(19) United States
(12) Patent Application Publication
(10) Pub. No.: US 2022/0151741 A1
(43) Pub. Date: May 19, 2022

(54) OSTEOGENIC POSTS FOR DENTAL IMPLANTS

(71) Applicant: OSTEOVANTAGE, INC., Fayetteville, AR (US)

(72) Inventors: Eric C. LEUTHARDT, Saint Louis, MO (US); Daniel W. MORAN, Ballwin, MO (US); Matthew R. MacEWAN MacEWAN, Saint Louis, MO (US)

(21) Appl. No.: 16/490,389

(22) PCT Filed: Mar. 2, 2018
(86) PCT No.: PCT/US2018/020674

§ 371 Date: Aug. 30, 2019

Related U.S. Application Data
(60) Provisional application No. 62/466,233, filed on Mar. 2, 2017.

(51) Int. Cl. A61C 8/02; A61N 1/32; A61N 1/05; A61N 1/02; A61C 8/00; A61C 13/225

(52) U.S. Cl. CPC A61C 8/0007; A61N 1/326; A61C 13/225; A61N 1/025; A61C 8/0028; A61N 1/0548

(57) ABSTRACT

Systems and methods are provided for enhancing osseointegration performed in conjunction with placing a dental implant. A dental implant post can be used for applying current to the implant site as well as for anchoring an artificial tooth. Such a dual purpose implant can allow faster healing and osseointegration time as well as enabling the use of dental implants in a large segment of the population currently disqualified from such treatment due to poor bone quality.

### INCORPORATION BY REFERENCE

All publications and patent applications mentioned in this specification are incorporated herein by reference in their entirety to the same extent as if each individual publication or patent application was specifically and individually indicated to be incorporated by reference.

### FIELD

This application relates to osteogenic dental implants and treatment.

### BACKGROUND

Over 240 million persons in the industrialized world (US, Europe, Japan) lack teeth. In the western world, 40% of the population is missing at least one tooth and 6-10% of the world's population is edentulous (having no teeth in either jaw). In the U.S., 11% are fully edentulous and 40% are partly edentulous. Of the approximately 240 million persons missing one or more teeth in the industrialized world, it is estimated that approximately 77% receive no treatment, 21% receive non-implant treatments and 2% (about 5.0 million) are treated with dental implants. It is estimated that approximately 1.5 million dental implants were performed in the U.S. during 2013.

Dental implants are titanium screw-like devices implanted into the mandible or maxilla to replace missing teeth. The process and components, including an implant, abutment, an artificial tooth or crown, are shown in FIGS. 1A and 1B. These implants play a critical role in restoring oral structure and dental function in edentulous patients by providing a stable, long-lasting support for functional prosthetic teeth. Dental implants also offer a more permanent solution than do dentures or bridgework.

Dental implant surgery is usually an outpatient surgery performed in stages, as shown in FIG. 1B. First, the damaged tooth is removed. The jawbone is prepared for surgery, a process that may involve bone grafting. After the patients jawbone heals, the oral surgeon places the dental implant metal post in the patient's jawbone. The patient then goes through a healing period that may last several months. The oral surgeon then places the abutment, which is an extension of the implant metal post. In some cases, when the implant is very stable, placing the abutment can be done at the same time that the implant is placed. After the soft tissue heals, the dentist will make molds of the patient's teeth and jawbone and later place the final tooth or teeth. The entire process can take many months from start to finish. Much of that time is devoted to healing and waiting for the growth of new bone in the patients jaw.

During surgery to place the dental implant, the oral surgeon makes a cut to open the patients gum and expose the bone. Holes are drilled into the bone where the dental implant metal post will be placed. Since the post will serve as the tooth root, it is implanted deep into the bone. At this point, the patient will still have a gap where the patient tooth is missing. A type of partial, temporary denture can be placed for appearance, if needed. The patient can remove this denture for cleaning and while the patient sleeps.

Once the metal implant post is placed in the patient's jawbone, osseointegration begins. During this process, the jawbone grows into and unites with the surface of the dental implant. This process, which can take several months, helps provide a solid base for the patient new artificial tooth—just as roots do for the patient's natural teeth. When osseointegration is complete, the patient commonly receives an additional surgery to place the abutment on the implant. The abutment is the piece where the crown will eventually attach. This minor surgery is typically done with local anesthesia in an outpatient setting. To place the abutment, the oral surgeon reopens the patients gum to expose the dental implant. The abutment is attached to the dental implant. The gum tissue is then closed around, but not over, the abutment. In some cases, the abutment is attached to the dental implant metal post when the post is implanted. That means the patient will not need an extra surgical step. Because the abutment juts past the gum line, however, it is visible when the patient opens their mouth, and will be that way until the patient's dentist completes the tooth prosthesis. Some people do not like that appearance and prefer to have the abutment placed in a separate procedure.

After the abutment is placed, the patient's gums must heal for one or two weeks before the artificial tooth can be attached. Once the patient gums heal, the patient will have more impressions made of their mouth and remaining teeth. These impressions are used to make the crown—the patient's realistic-looking artificial tooth. The crown can't be placed until the patient's jawbone is strong enough to support use of the new tooth. The dental specialist can choose artificial teeth that are either removable, fixed or a combination of both.

Unfortunately, up to 25% of patients are not candidates for dental implants due to lack of sufficient bone in the alveolar ridge needed to support an implant. This prevents successful instrumentation of metallic implants, leading to reduced anterior facial height, undesirable cosmetic effects, and poor quality of life. Surgical reconstruction and repair of a deficient alveolar ridge (aka. alveolar ridge augmentation) thereby represents a critical step in many oral surgeries and prosthetic solutions.

However, due to the limitation of existing procedures, oral surgeons commonly employ surgical adjuncts capable of enhancing bone growth at the time of augmentation surgery. While some techniques are available, improvement in the field of dental osetogenesis is needed.

### SUMMARY OF THE DISCLOSURE

In an aspect of the invention, embodiments of osteogenic dental implant systems are provided. The systems comprise an osteogenic screw configured for implant into a patient's jawbone, the screw being selectively anodized to form an electrically conductive first portion and an electrically insulated second portion, the electrically insulated second portion comprising about 75% of the surface of the screw; an electrical connector at a top portion of the screw and configured to transmit power to the osteogenic screw; and a removable power source operatively connected to the electrical connector.

In some embodiments, the electrically insulated portion comprises about 75% of the surface of the screw. In other embodiments, the electrically insulated portion is only 25% of the surface of the screw. The electrically conductive first portion can comprise a titanium alloy. Other materials are also possible. In some embodiments, the electrically insulated second portion comprises a titanium dioxide. Other materials are also possible. The electrically conductive first portion can be on a threaded portion of the screw. In some embodiments, the electrically insulated second portion is on a thread portion of the screw. The removable power source can comprise a temporary crown, an artificial tooth, or an abutment. In some embodiments, the removable power source comprises a return electrode. The removable power source can comprise a retainer that can be worn and removed by the patient. In some embodiments, the retainer comprises a return electrode facing an inner portion of the patient's cheek. A large portion 75% of the retainer can be electrically conductive. In some embodiments, the retainer comprises at least one of logic, sensors, and memory. The retainer can be rechargeable. In some embodiments, the power source is removed after osseointegration has occurred. The power source can be worn continually until osseointegration has occurred. In some embodiments, the power source is repeatedly removed and re-applied until osseointegration has occurred.

In another aspect, embodiments of a method of alveolar ridge preparation are provided. The method comprises implanting a dental implant into the jawbone of a patient, the implant comprising an osteogenic screw configured for implant into a patient's jawbone, the screw being selectively anodized to form an electrically conductive first portion and an electrically insulated second portion, the electrically insulated second portion comprising about 75% of the surface of the screw and an electrical connector at a top portion of the screw configured to transmit power to the osteogenic screw; operatively connecting the electrical connector to a removable power source; and applying an electrical current for a treatment duration and treatment period such that osseointegration sufficient for placement of at least one of an abutment, artificial tooth, or crown has occurred.

In some embodiments, the electrical current is about 1 μA-1 mA. The treatment duration can comprise continuous treatment. In some embodiments, the treatment period is about 3 months. The method can further comprise assessing the patient for eligibility for treatment. In some embodiments, the method further comprises confirming sufficient osseointegration has occurred. The method can further comprise placing at least one of an abutment, an artificial tooth, and a crown.

In yet another aspect, embodiments of an osteogenic dental implant system are provided. The system comprises an osteogenic screw configured for implant into a patient's jawbone, the screw being selectively anodized to form an electrically conductive first portion and an electrically insulated second portion, the electrically insulated second portion comprising about 75% of the surface of the screw; and a dual purpose socket positioned at the top of the screw, the socket configured to attach to a removable electrical connector and attach to a mating mechanism for at least one of an abutment, an artificial tooth and a crown.

In some embodiments, the system further comprises an electrical connector attached to the socket. The system can further comprise a mating mechanism attached to the socket. In some embodiments, the electrically insulated portion comprises about 75% of the surface of the screw. The electrically conductive first portion can comprise a titanium alloy. In some embodiments, the electrically insulated second portion comprises a titanium dioxide. The electrically conductive first portion can be on a threaded portion of the screw. In some embodiments, the electrically insulated second portion is on a thread portion of the screw. The removable power source can comprise a temporary crown, an artificial tooth, or an abutment. In some embodiments, the removable power source comprises a return electrode. The removable power source can comprise a retainer that can be worn and removed by the patient. In some embodiments, the retainer comprises a return electrode facing an inner portion of the patient's cheek. A large portion (e.g., 75%) of the retainer can be electrically conductive. In some embodiments, the retainer comprises at least one of logic, sensors, and memory. The retainer can be rechargeable. In some embodiments, the power source is removed after osseointegration has occurred. The power source can be worn continually until osseointegration has occurred. In some embodiments, the power source is repeatedly removed and re-applied until osseointegration has occurred.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A and 1B show a typical process and components for dental implant surgery.

FIGS. 2A-2C depict various embodiments of a dental implant.

FIG. 3 illustrates embodiments of a dental implant and power source.

FIG. 4 shows embodiments of a dental implant and power source.

FIG. 5 illustrates an embodiment of a method of treatment.

FIGS. 6A-6C depict embodiments of varying implant sizes and suitability.

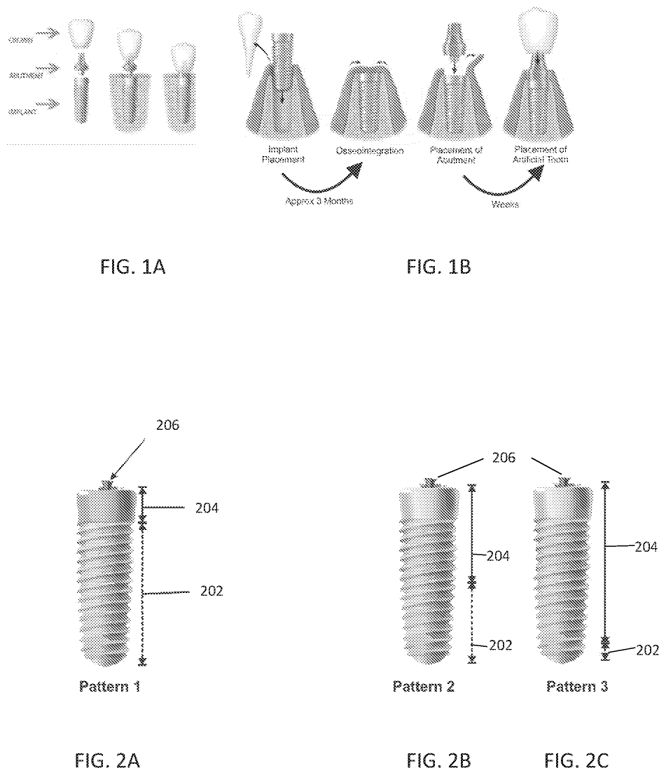

### DETAILED DESCRIPTION

Accelerating the time for osseointegration in which the bones grow around the dental implant would be clinically very important. This will lessen the time that the patient needs to wait for the final artificial tooth can be attached. In addition to accelerating the rate of bone growth, enhancing the degree to which bone grows can also lessen the risk of an implant failure, or enable implant placement in patients who have poor bone quality, thinned jaw bone, or bone growth risk factors, potentially allowing dental implants in up to 25% of the population now disqualified from receiving them.

Bone growth can be stimulated by various means. In the setting of spinal surgery, direct electrical current is applied to the electrodes to stimulate bone growth and fuse the fragments and adjoining vertebrae. To permit the current to be applied for extended periods of time while permitting the patient to be mobile, a generator is connected to the wire electrodes and implanted between the skin and muscle near the patient's vertebral column. The generator provides a continuous low amperage direct current (e.g., 40 μA) for an extended period of time (e.g., six months). After the vertebrae are fused, the generator and leads are surgically removed.

In the setting of dental implants, this invention uses a metal implant, or post, which screws into the jawbone as both a mechanical stabilizing device and a conduit for electrical energy to induce osteogenesis. This dual role is accomplished by having portions of the screw be conductive and other portions being non-conductive to enable conformal and specified regions to be deliberately stimulated while others anatomic locations are avoided.

For bony implants, screws are mounted in the hone or bones being fused. Although these screws work well for their intended purpose, they do not facilitate electrical stimulation the region. Moreover, if electrical stimulation were applied to bones having conventional screws, the screws could potentially conduct current to areas of tissue and bone where the current is unneeded and where the current could potentially have adverse effects. Thus, there are drawbacks and potential problems associated with conventional screws bein used to conduct current to induce bone growth.

On one aspect, a system for use in stimulating at least one of bone growth control in a patient generally comprises a screw and an electrode. The first screw has an elongate shaft with opposite ends and a length extending between the ends, an exterior surface and a screw thread formed on the exterior surface of the shaft and extending along at least a portion of the length. The screw thread has an electrically conducting portion and an electrically insulating portion. FIGS. 2A-2C depict various embodiments of a screw, comprising varying patterns of conducting and insulating portions. As will be appreciated by those skilled in the art, the screw comprises an electrically conductive material 202 such as a titanium alloy and the electrically insulating portion of the shaft 204 is coated with an insulating material such as titanium dioxide. Other materials are also possible. The electrically conducting portion 202 is located for deposition in a first pre-specified portion of the patient's anatomy (boney portion of the jaw) and the electrically insulating portion 204 is located for deposition in a second pre-specified portion of the patient (the non-bony portion of the jaw and gums). An electrical power source is adapted to pass electrical current through the patient between the first screw and second electrode. The electrical power source is operatively connected to the first screw through a connector 206 positioned at the top of the screw that is electrically insulated for selectively conveying current through the electrically conducting portion 202 of the screw thread to the first pre-specified portion. The electrically insulating portion 204 inhibits current from being conveyed to or released from the second pre-specified portion. Notably there can be different patterns of the insulated portion 204 of the screw to optimize the electric fields for the intended purpose of bone growth, healing, and pain control. The screw can be selectively anodized to form titanium dioxide on most of its surface, as shown in FIG. 2C (e.g. about >50% coverage, 60% coverage, 75% coverage, 80% coverage, 90% coverage, 60-75% coverage, 70-90% coverage, or more) such that the deepest portion of the jaw grows bone while reducing the current density passing through the gums or non-bony portion of the jaw. Alternatively, a lesser amount of the screw can be anodized, as shown in FIG. 2A (e.g. about 10%, 25%, 35%, 45%, 10-25%, 15-35%, 25-45%) to optimize that the maximal amount of the bony jaw gets exposed to osteoinductive electrical fields. FIG. 2B shown an embodiment in which closer to half of the amount of the screw is anodized.

In some embodiments, the screw comprises a dual-purpose socket at the top of the screw. The socket can be configured to be attached to an electrically insulated connector to transmit power from a power source. Portions of the socket can be electrically conductive to transmit power from the connector. The socket can also be configured to be attached to a mating mechanism for mating with at least one of an abutment, artificial tooth, and a crown.

In some embodiments, the anodization pattern can comprise a thickness of about 1-1000 nm (e.g., about 0-250 nm, 250-500 nm, 500-750 nm, 750-1000 nm, 0-500 nm, 500-1000 nm, etc.). The anodization can be Type I, Type II, or other types of anodization.

In some embodiments, the anodization pattern can comprise alternating regions of anodization. For example, the pattern can comprise a striped pattern, each stripe extending around a circumference of the implant. In some embodiments, the stripes can extend along a length of the implant. In some embodiments, an anodization portion extends around an entire circumference of the implant. In some embodiments, an anodization pattern extends around less than an entire circumference of the implant. In other embodiments the anodization pattern can be graded across the screw, such that the thickness of anodization changes in linear gradation along the length of the screw or in an exponential gradation of thickness along the length of the screw. Alternatively, the insulated and uninsulated patterns can alternate in either rings or dots to modify the electrical field to optimize conformance to the adjacent bone.

In some embodiments, a width of the implant is about 3-6 mm. Other widths are also possible (e.g., about 3 mm, 3.5 mm, 4 mm, 5 mm, 6 mm, 3.5-5 mm, etc.). In some embodiments, a length of the implant is about 5-11 mm. Other lengths are also possible (e.g., about 5 mm, 6, mm, 7 mm, 8 mm, 9 mm, 10 mm, 11 mm, 5-10 mm, 6-10 mm, 6-8 mm, 5-8 mm, etc.)

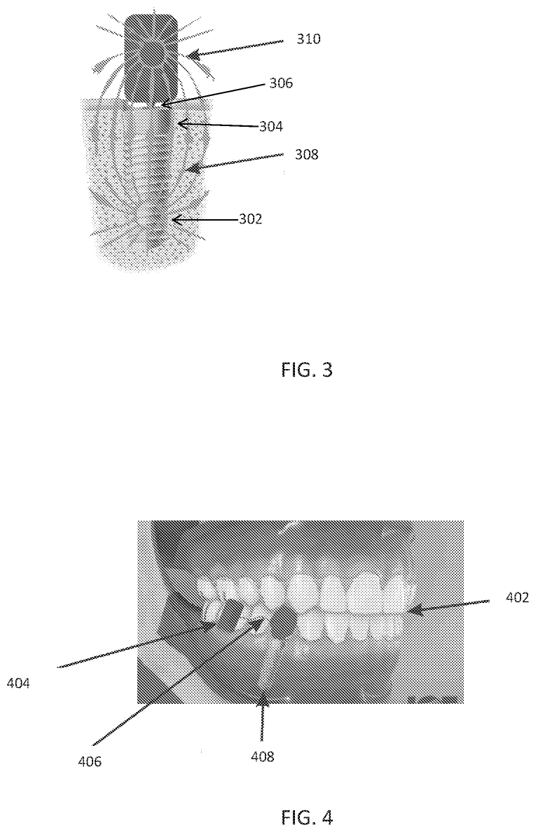

The power source 310 can be physically mounted onto the top of the screw that has both conducting 302 and insulating 304 portions, as shown in FIG. 3. The power source 310 can be connected to connector 306 portion of the screw. This power source 310 can be in the form of a sealed battery and include circuitry and other components (e.g, telemetry, wireless connectivity, control, etc.). This battery can be further encased in the form of a temporary crown or artificial tooth, or abutment. This power source 310 can be removable such that once the osseointegration has occurred, the power source 310 can be removed and replaced by a permanently attached artificial tooth. Alternatively, it can be replaced by a detachable tooth. In operation, an electrical stimulus is generated using power source 310 that induces an osteogenic field in the implant, as shown in FIG. 3.

Turning to FIG. 4, the power source (and associated circuitry) 404 can be part of an easily removed dental apparatus such as a retainer 402, or other temporary orthodontic device, as shown in FIG. 4. The retainer can comprise an apparatus shaped to conform to one or more teeth of a patient. The retainer can be configured to conform to one or more upper teeth, lower teeth, or both. The retainer can comprise a wired retainer or a polymer based retainer (e.g., clear retainer). Thus, the power source and return electrode (e.g. anode) can be within the retainer and can electrically couple with the osteogenic screw/post 408 when worn by the patient, for example through wire connection 406. This use configuration would allow the battery in the retainer to be recharged when not being actively used by the patient and could allow for longer stimulation durations nearly independent of the charge of a single battery since recharge is readily available.

In some embodiments (e.g. power source 404, power source 310), a longer term affixed battery/power source and corresponding stimulation patterns are possible. There are also intermediate variations that combine these approaches. An example of this would include a battery that is attached to the implant, but is inductively recharged through capacitive coupling. Thus, the patient would wear a retainer during the evening in which the power would provide a dual function in which the some power is used for delivering osteogenic current to the mandible of the jaw and some power is used to recharge the battery that is affixed to the implant in the bony jaw. Thus, for example, when the retainer is not in the mouth the battery affixed to the implant would provide enough power to stimulate bone growth until the retainer is returned to the mouth. In some embodiments, a power supply (e.g., within an abutment, artificial tooth, retainer, etc.) comprise wireless receiver configured to transduce a wireless power signal from an external transmitter worn by the patient. In this matter, power can be wireless delivered to a power supply without the use of a battery.

In regards to the second (return) electrode component of the dental osteogenic system, this electrode can be in several configurations. The second electrode can be part of the power source that is mechanically and electrically affixed to the osteogenic post. This may not be optimal due to the proximity of return electrode being too close to the region of desired osteogenesis. Proximity can also lead to bony alterations of adjacent teeth. Alternatively, the return electrode could be part of the removable retainer. This could provide a potential advantage in that the electrode could be made to face the inner cheek and any osteomodulatory effects that the return electrode could have on the adjacent teeth would be mitigated. Another variation is that the a large portion of the retainer (e.g., about 75%, 50-75%, greater than 75%) or the entire retainer, is conductive such that the currents in the return electrode are widely distributed thus reducing current density around the return electrode and further limiting the impact of the electrical circuit has on non-targeted tissues (versus the pro-osteogenic cathodic current delivered to the jaw bone). Yet another variation of the retainer is that the electrode configuration can be explicitly constructed such that the current can be steered to an optimal site within the jaw. Thus depending on which tooth is being replaced and what the position of the implant is the electrode location/configuration can be altered to be patient specific and optimized for maximal bone deposition. Another iteration could include multiple return electrodes being present throughout that could be configured at the beginning for usage throughout the treatment regime to optimize or alter bone deposition.

In addition to the benefits of having the power source and return electrode in the temporarily worn retainer there are other additional technical advantages. Specifically, the circuitry embedded in the retainer can contain logic, sensors, and memory to perform a number of additional computations that could be clinically useful. Examples where having an elevated degree of “intelligence” associated with the retainer could include monitoring and registering usage information (how often was the retainer worn), indicating the power levels of the battery, monitoring and registering the electric changes occurring with the osteogenic stimulation that could indicate bony changes (e.g., electrical impedance of the system), to name a few. Additionally the circuitry could contain wireless, or wired, communication such that the information could be transmitted in various formats, such as Bluetooth transmission, or direct wired downloads when taken out of the mouth.

Collectively, one skilled in the arts can envision a user scenario in which the osteogenic implant/screw with conductive (in the boney mandible) and insulated portions (adjacent to the non-boney jaw and gums) are surgically placed in the jaw. The patient is instructed to wear a retainer on, for example, a nightly basis. The retainer contains power, battery, circuitry, logic, memory, and sensors. While wearing the retainer electrical current (e.g., 1-100 μA DC electric current) is drawn into the osteogenic screw. The retainer can either provide this current by a direct connection the screw implant or wirelessly through an inductive current that connects to a battery/circuitry that is attached to the screw implant. During the day, the retainer is removed and placed on a base that enables recharging of the battery and download of any data acquired while the patient was wearing the retainer. The downloaded information can then be provided via internet to the patient or to the care providers or third parties. This communication can also let care providers and third parties alter the parameters of stimulation depending on clinical course. Examples could include increasing or decreasing the amperage of stimulation, or altering which anodes are used in the retainer to change bone deposition pattern.

In some embodiments, the retainer can comprise both the anode and the cathode and be used without the osteogenic screw. In such embodiments, the retainer itself is used to promote osteogenesis. In such embodiments, the electrically conductive material is selectively positioned to treat appropriate regions.

In the past prior inventions have taught the use of making an osteoinductive implant out of titanium (e.g., U.S. Pat. Nos. 8,380,319B2, 4,027,392A, 8,374,697B2, 5,738,521A, 5,725,377A, 5,292,252A). These past inventions do not, however, discuss the use of anodization, such as titanium dioxide, as a method to enable an insulating and a non-insulating portion of the implant. US20120276501A1 does teach that anodization of the titanium can be used as an insulating separator. They do not, however, teach that this anodization can be patterned in such a way to optimize cathodic current being preferentially being deposited in the bony portion of the mandible. They specifically state the separator is “preferably of a minimal shape and size to ensure electrical isolation. This is distinct from the proposed optimal pattern of this implant where the majority of the implant is anodized to avoid current shunting through non-boney tissues. Furthermore, when considered in the context of the retainer as the source of power and location of the circuitry this is physically separable construct, which also facilitates an anode that is more amenable to steering current to the most appropriate anatomic location. Also US20120276501A1 teaches that the waveforms of the electrical current provided would be in the form of alternating current (AC) or pulsed square waves, versus direct current (DC). In this setting, alternating current would capacitively couple (i.e. shunt) current through a titanium dioxide layer, thus rendering the titanium dioxide layer useless as something that blocks or steers currents through the implant. Furthermore, studies have shown that DC stimulation, not AC stimulation, promotes bone formation.

It will be appreciated that the system described herein can use a combination of hardware, software, and/or firmware implementations of aspects of the system. The specific configuration can be selected taking into consideration cost vs. efficiency tradeoffs. There are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation. In other embodiments, the implementer may opt for some combination of hardware, software, and/or firmware. Hence, there are several possible vehicles by which the processes and/or devices and/or other technologies described herein can be effected, dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

In some implementations described herein, control structures can be used to implement methods described herein. For example, logic and similar implementations can include software or other control structures. Electronic circuitry, for example, may have one or more paths of electrical current constructed and arranged to implement various functions as described herein. In some implementations, one or more media can be configured to bear a device-detectable implementation when such media hold or transmit a device detectable instructions operable to perform as described herein. In some variants, for example, implementations can include an update or modification of existing software or firmware, or of gate arrays or programmable hardware, such as by performing a reception of or a transmission of one or more instructions in relation to one or more operations described herein. Alternatively or additionally, in some variants, an implementation can include special-purpose hardware, software, firmware components, and/or general-purpose components executing or otherwise invoking special-purpose components. Specifications or other implementations can be transmitted by one or more instances of tangible transmission media as described herein, optionally by packet transmission or otherwise by passing through distributed media at various times.

Alternatively or additionally, implementations may include executing a special-purpose instruction sequence or otherwise invoking circuitry for enabling, triggering, coordinating, requesting, or otherwise causing one or more occurrences of any functional operations described above. In some variants, operational or other logical descriptions herein may be expressed directly as source code and compiled or otherwise invoked as an executable instruction sequence. In some contexts, for example, C++ or other code sequences can be compiled directly or otherwise implemented in high-level descriptor languages (e.g., a logic-synthesizable language, a hardware description language, a hardware design simulation, and/or other such similar mode(s) of expression). Alternatively or additionally, some or all of the logical expression may be manifested as a Verilog-type hardware description or other circuitry model before physical implementation in hardware, especially for basic operations or timing-critical applications. Those skilled in the art will recognize how to obtain, configure, and optimize suitable transmission or computational elements, material supplies, actuators, or other common structures in light of these teachings.

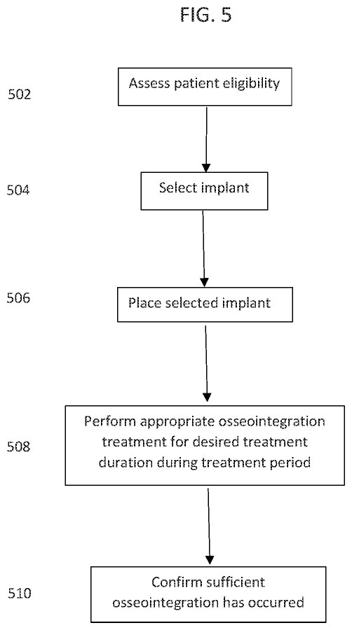

As shown in FIG. 5, a method for implanting the systems described herein can comprise first assessing patient eligibility, shown at step 502. Once it has been determined that a patient is eligible, a suitable implant is selected, shown at step 504. The implant can then be placed in the patient's jawbone, shown at step 506. Appropriate osseointegration treatment is performed for the desired treatment duration and for a desired treatment period, shown at step 508. After treatment, a clinician can confirm that sufficient osseointegration has occurred, shown at step 510. If sufficient osseointegration has not occurred, further treatment can be performed. If sufficient treatment has been performed, an abutment, crown, and/or artificial tooth can be subsequently placed. More details surrounding this method are provided below.

Factors that can affect eligibility include poor bone quality or medical comorbidities inhibiting bone growth and suboptimal anatomy (e.g. too-thin mandible). Poor bone quality can be defined and evaluated by various Xray techniques (e.g., plain films, CT scans, or bone density scans). Medical comorbidities include diabetes, smoking/tobacco usage, cancer, poor nutrition, osteopenia, osteoporosis, chronic steroid usage, bad periodontal disease, chemotherapy, radiation therapy, patients undergoing immunosuppression, bone and blood disorders, bone marrow cancer, parathyroid disorders

Traditionally, patients presenting with the bone qualities or comorbidities described above would be disqualified from receiving a dental implant. However, the inventions described herein can be used to enhance the bone in a manner sufficient to allow such patients to undergo the dental implant procedure. A patient can be deemed eligible to undergo the procedure once the jawbone can provide adequate support. Since dental implants fuse with the jawbone in order to anchor the tooth, adequate bone density and quality is one of the most important requirements for dental implant success. Bone grafts or mini implants are sometimes considered for patients who do not have enough bone support.

Other important factors to consider in determining patient eligibility are good overall health, healthy gums, commitment to oral health, and absence of teeth clenching. Healthy gums are necessary to support the new dental implant as it fuses with the jawbone. Patients with a high risk of periodontal (gum) disease often experience dental implant failure. A dental implant requires excellent dental hygiene. A commitment to daily brushing and flossing, along with periodic visits to the dentist is crucial. A patient struggling with bruxism or teeth clenching can be encouraged to visit with your dental specialist as such a habit has been shown to decrease success rates of implant dentistry. X rays, dental examinations, and medical histories can be used in assessment of patient eligibility.

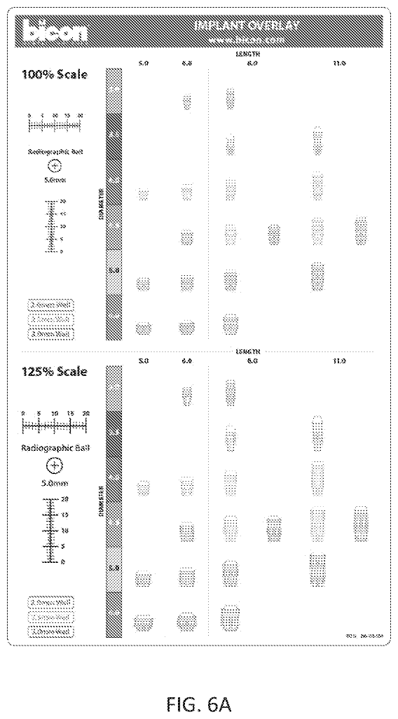

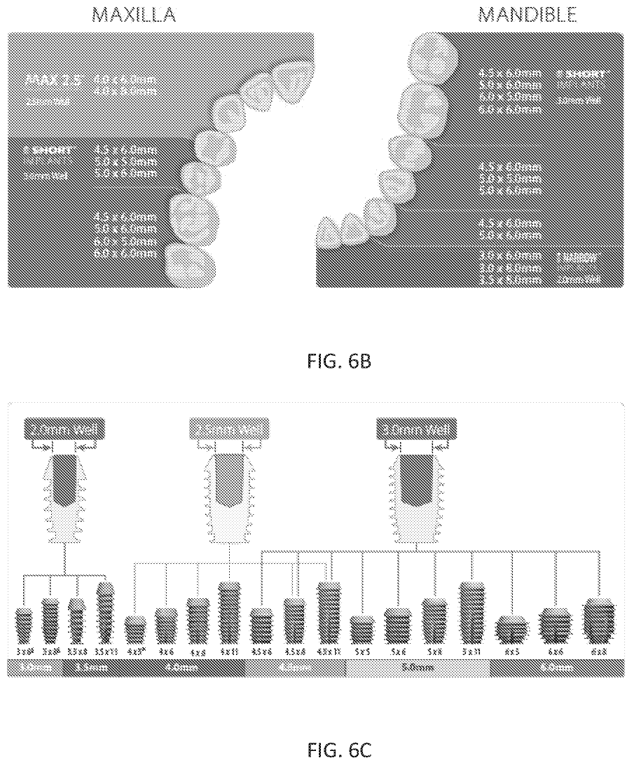

Once a dental specialist determines that a patient is eligible for the procedure described herein, an appropriate implant is selected. An implant with a diameter of about 3.5 mm is generally used for mandibular anterior teeth. If practical (but not always), the use of such an implant is avoided for maxillary anterior and all posterior teeth. From the canine posteriorly, one implant is generally placed per tooth being replaced, when practical. It can be preferred to have at least about 1.0 mm of bone around the implant. The width of the alveolar bone can be assessed with a periodontal probe or a caliper. Therefore, an appropriate bone width can be about 5.5 mm to comfortable accommodate an about 3.5 mm implant, unless ridge splitting or grafting techniques are employed to widen the implant site. In the anterior maxilla, it can be preferred to place implants such as the MAX 2.5™ implants. In this area, there may be a need for ridge splitting or bone grafting techniques. FIGS. 6A-6C provide guidance as to suitable implant selection. The implants shown in FIGS. 6A-6C can comprise any combination of features described above, for example, with reference to FIGS. 2A-3.

During the implant surgery, the surgeon makes a cut to open the gum and expose the bone. The surgeon drills a hole into the bone about 4-12 mm deep in the location the implant post will be placed. Other depths are also possible (about 5-11 mm, about 5-10 mm, about 5-9 mm, about 5-8 mm, about 6-7 mm, about 5 mm, about 6 mm, about 7 mm, about 8 mm, or longer). The implant is placed so that it extends about 4-12 mm into the bone. Other depths are also possible (e.g., about 5-11 mm, about 5-10 mm, about 5-9 mm, about 5-8 mm, about 6-7 mm, about 5 mm, about 6 mm, about 7 mm, about 8 mm, or longer).

After implantation of the post, the osseointegration process can occur. Direct current can be applied to the post. In some embodiments, between about 1 μA and 5 mA is applied. In some embodiments, direct current of about 20 μA-60 μA is applied. In some embodiments, between about 1 μA and 1 mA is applied. In some embodiments, the current can be any time-varying current waveform (e.g., a sine wave, a square wave) with a frequency between about 0-10 GHz.

The energy can be applied continuously during the treatment period. In some embodiments, the treatment is episodic, ranging from about 30 minutes to 23 hours per day during the treatment period. In some embodiments, the treatment period is about 3 months. Other treatment periods are also possible (e.g., about 1 week-6 months, 1 month, 2 months, 4 months, 5 months, 6 months, 3-6 months, 1-3 months, 2-4 months, 2-6 months, 4-6 months, or longer).

The treatment regimen can be adjustable by the clinician based on the specific patient's needs. For example, the treatment duration, treatment period, amplitude, and location of the stimulus could be controlled and customized and adjusted over time or in response to clinical evaluation.

After an initial treatment period, a clinician can check to see whether sufficient osseointegration has occurred for placing. This can be verified using X Rays, CT scans, or bone density scans. If sufficient osseointegration has not occurred, the treatment period may be extended and the treatment re-introduced. Once the clinician is satisfied that sufficient osseointegration has taken place, in cases in which an abutment was not previously placed with the implant, they may perform surgery to place the abutment. The gum is reopened to attach the abutment to the dental implant. After placement of the abutment, the gums must heal for about 1-2 weeks before the artificial tooth is attached. This healing time may be reduced using the energy application methods described herein.

The implant system described herein can dramatically improve the clinical success of vertical alveolar ridge augmentation, reduce the risk of graft resorption, improve quality of life post-operatively, and reduce the growing cost of repeat oral surgery. Specifically, the osteogenic dental implant can dramatically improve the success of contemporary bone grafting techniques. By inducing focal bone formation and preservation in critical areas of interest, the novel implant reduces the incidence of failed augmentation and, thereby, the risk of costly secondary surgeries and procedures. Preliminary animal data suggest that the implants described herein can double the rate of bone formation, which translates to more successful graft incorporation, and potentially a 5X reduction in the risk of failed augmentation. Improvement in the overall success of oral surgery translates to dramatic reductions in post-operative complications and debilitating side effects. Improved alveolar augmentation is largely anticipated to reduce the incidence of post-operative pain, increase post-operative function, and improve quality of life. Subsequently, the dental implant system is targeted at eliminating >80% of secondary surgical procedures and reducing the need for more complex surgical interventions.

In still further alternatives, an embodiment of the implant system described herein may be adopted and configured for use in combination with autologous and/or xenogenic bone graft materials on other biomolecular materials. The combination may be selected based on expected interactions or changes to the osteogenic process as a result of a reaction to the electrical signals generated by the system.

Autologous and xenogenic bone graft materials are commonly enhanced through the addition of recombinant human bone morphogenetic protein-2 (rhBMP-2), rhBMP-4, rhBMP-7, or recombinant human platelet-derived growth factor-BB (rhPDGF-BB). BMP and PDGF harness natural biomolecular mechanisms to accelerate local osteogenesis and, despite varying indications of use, are commonly utilized in spinal fusion procedures off-label. Unfortunately, utilization of osteobiologic adjuncts presents additional risks and limitations. The use of BMP implants remains controversial due to high rates of adverse events (e.g., 10-50%) such as excessive inflammation, swelling, and airway closure. The prohibitive cost (e.g., Prospec Protein Specialists; $4682/1 mg rhBMP-2 for laboratory use) also limits adoption. Non-pharmaceutical adjuncts have therefore gained significant traction as an alternate means of inducing new bone formation without the complication and cost of BMP implants. Direct current electrical stimulation (DC, DCES), commonly utilized in long bone fusion, offers a unique and proven method of inducing and controlling new bone formation. Yet, existing bone growth stimulators are significantly limited by methods of topical application and the bulky nature of the associated power supply. Deployment of existing DC stimulators therefore requires a two stage procedure involving separate implantation of graft material and stimulator leads/implantable battery pack; significantly increasing the complexity and length of surgery. As a result, the present form factor and design of existing DC stimulators has limited clinical adoption by disrupting existing surgical work flow, increasing procedural complexity, and raising intraoperative risk.

Additionally or optionally, embodiments of the methods and systems described herein may be advantageously used to reduce or prevent micro-gap formation, bacterial ingress, and marginal bone loss/resorption at the interface between the metallic implant (i.e. post) and the bone.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein can be implemented via Application Specific Integrated Circuits (ASICs), Field Programmable Gate Arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution.

In a general sense, those skilled in the art will recognize that the various embodiments described herein can be implemented, individually and/or collectively, by various types of electro-mechanical systems having a wide range of electrical components such as hardware, software, firmware, and/or virtually any combination thereof and a wide range of components that may impart mechanical force or motion such as rigid bodies, spring or torsional bodies, hydraulics, electro-magnetically actuated devices, and/or virtually any combination thereof. Consequently, as used herein “electro-mechanical system” includes, but is not limited to, electrical circuitry operably coupled with a transducer (e.g., an actuator, a motor, a piezoelectric crystal, a Micro Electro Mechanical System (MEMS), etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, communications switch, optical-electrical equipment, etc.), and/or any non-electrical analog thereto, such as optical or other analogs. Those skilled in the art will also appreciate that examples of electro-mechanical systems include but are not limited to a variety of consumer electronics systems, medical devices, as well as other systems such as motorized transport systems, factory automation systems, security systems, and/or communication/computing systems. Those skilled in the art will recognize that electro-mechanical as used herein is not necessarily limited to a system that has both electrical and mechanical actuation except as context may dictate otherwise.

In a general sense, the various aspects described herein can be implemented, individually and/or collectively, by a wide range of hardware, software, firmware, and/or any combination thereof and can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), and/or electrical circuitry forming a communications device (e.g., a modem, communications switch, optical-electrical equipment, etc.). The subject matter described herein can be implemented in an analog or digital fashion or some combination thereof.

Those skilled in the art will recognize that at least a portion of the systems and/or processes described herein can be integrated into an image processing system. A typical image processing system generally includes one or more of a system unit housing, a video display device, memory such as volatile or non-volatile memory, processors such as microprocessors or digital signal processors, computational entities such as operating systems, drivers, applications programs, one or more interaction devices (e.g., a touch pad, a touch screen, an antenna, etc.), control systems including feedback loops and control motors (e.g., feedback for sensing lens position and/or velocity; control motors for moving/distorting lenses to give desired focuses). An image processing system can be implemented utilizing suitable commercially available components, such as those typically found in digital still systems and/or digital motion systems.

Those skilled in the art will recognize that at least a portion of the systems and/or processes described herein can be integrated into a data processing system. A data processing system generally includes one or more of a system unit housing, a video display device, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for sensing position and/or velocity; control motors for moving and/or adjusting components and/or quantities). A data processing system can be implemented utilizing suitable commercially available components, such as those typically found in data computing/communication and/or network computing/communication systems.

Those skilled in the art will recognize that at least a portion of the systems and/or processes described herein can be integrated into a mote system. Those having skill in the art will recognize that a typical mote system generally includes one or more memories such as volatile or non-volatile memories, processors such as microprocessors or digital signal processors, computational entities such as operating systems, user interfaces, drivers, sensors, actuators, applications programs, one or more interaction devices (e.g., an antenna USB ports, acoustic ports, etc.), control systems including feedback loops and control motors (e.g., feedback for sensing or estimating position and/or velocity; control motors for moving and/or adjusting components and/or quantities). A mote system may be implemented utilizing suitable components, such as those found in mote computing/communication systems. Specific examples of such components entail such as Intel Corporation's and/or Crossbow Corporation's mote components and supporting hardware, software, and/or firmware.

In certain cases, use of a system or method may occur in a territory even if components are located outside the territory. For example, in a distributed computing context, use of a distributed computing system may occur in a territory even though parts of the system may be located outside of the territory (e.g., relay, server, processor, signal-bearing medium, transmitting computer, receiving computer, etc. located outside the territory). A sale of a system or method may likewise occur in a territory even if components of the system or method are located and/or used outside the territory.

Further, implementation of at least part of a system for performing a method in one territory does not preclude use of the system in another territory.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “operably coupled to” each other such that the desired functionality is achieved, irrespective of architectures or intermedial components Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wireles sly interactable, and/or wirelessly interacting components, and/or logically interacting, and/or logically interactable components.

In some instances, one or more components can be referred to herein as “configured to,” “configured by,” “configurable to,” “operable/operative to,” “adapted/adaptable,” “able to,” “conformable/conformed to,” etc. Those skilled in the art will recognize that such terms (e.g. “configured to”) can generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications can be made without departing from the subject matter described herein and its broader aspects and, therefore, the appended claims are to encompass within their scope all such changes and modifications as are within the true spirit and scope of the subject matter described herein.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

This disclosure has been made with reference to various example embodiments. However, those skilled in the art will recognize that changes and modifications may be made to the embodiments without departing from the scope of the present disclosure. For example, various operational steps, as well as components for carrying out operational steps, may be implemented in alternate ways depending upon the particular application or in consideration of any number of cost functions associated with the operation of the system; e.g., one or more of the steps may be deleted, modified, or combined with other steps.

Additionally, as will be appreciated by one of ordinary skill in the art, principles of the present disclosure, including components, may be reflected in a computer program product on a computer-readable storage medium having computer-readable program code means embodied in the storage medium. Any tangible, non-transitory computer-readable storage medium may be utilized, including magnetic storage devices (hard disks, floppy disks, and the like), optical storage devices (CD-ROMs, DVDs, Blu-ray discs, and the like), flash memory, and/or the like. These computer program instructions may be loaded onto a general purpose computer, special purpose computer, or other programmable data processing apparatus to produce a machine, such that the instructions that execute on the computer or other programmable data processing apparatus create a means for implementing the functions specified. These computer program instructions may also be stored in a computer-readable memory that can direct a computer or other programmable data processing apparatus to function in a particular manner, such that the instructions stored in the computer-readable memory produce an article of manufacture, including implementing means that implement the function specified. The computer program instructions may also be loaded onto a computer or other programmable data processing apparatus to cause a series of operational steps to be performed on the computer or other programmable apparatus to produce a computer-implemented process, such that the instructions that execute on the computer or other programmable apparatus provide steps for implementing the functions specified.

The foregoing specification has been described with reference to various embodiments. However, one of ordinary skill in the art will appreciate that various modifications and changes can be made without departing from the scope of the present disclosure. Accordingly, this disclosure is to be regarded in an illustrative rather than a restrictive sense, and all such modifications are intended to be included within the scope thereof. Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, a required, or an essential feature or element. As used herein, the terms “comprises,” “comprising,” and any other variation thereof are intended to cover a non-exclusive inclusion, such that a process, a method, an article, or an apparatus that comprises a list of elements does not include only those elements but may include other elements not expressly listed or inherent to such process, method, system, article, or apparatus.

In an embodiment, the system is integrated in such a manner that the system operates as a unique system configured specifically for function of the system for monitoring an individual subject and facilitating a motion regimen of the individual subject (e.g., system 1000), and any associated computing devices of the system operate as specific use computers for purposes of the claimed system, and not general use computers. In an embodiment, at least one associated computing device of the system operates as a specific use computer for purposes of the claimed system, and not a general use computer. In an embodiment, at least one of the associated computing devices of the system is hardwired with a specific ROM to instruct the at least one computing device. In an embodiment, one of skill in the art recognizes that the system for monitoring an individual subject and facilitating a motion regimen of the individual subject (e.g., system 1000) effects an improvement at least in the technological field of monitoring and effecting body movements.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. An osteogenic dental implant system, the system comprising:
an osteogenic screw configured for implant into a patient's jawbone, the screw being selectively anodized to form an electrically conductive first portion and an electrically insulated second portion, the electrically insulated second portion comprising about 75% of the surface of the screw;
an electrical connector at a top portion of the screw and configured to transmit power to the osteogenic screw; and
a removable power source operatively connected to the electrical connector.

2. The system of claim 1, wherein the electrically insulated portion comprises about 75% of the surface of the screw.

3. The system of claim 1, wherein the electrically conductive first portion comprises a titanium alloy.

4. The system of claim 1, wherein the electrically insulated second portion comprises a titanium dioxide.

5. The system of claim 1, wherein the electrically conductive first portion is on a thread portion of the screw.

6. The system of claim 1, wherein the electrically insulated second portion is on a thread portion of the screw.

7. The system of claim 1, wherein the removable power source comprises a temporary crown, an artificial tooth, or an abutment.

8. The system of claim 1, wherein the removable power source comprises a return electrode.

9. The system of claim 1, wherein the removable power source comprises a retainer that can be worn and removed by the patient.

10. The system of claim 9, wherein the retainer comprises a return electrode facing an inner portion of the patient's cheek.

11. The system of claim 9, wherein about 75% of the retainer is electrically conductive.

12. The system of claim 8, wherein the retainer comprises at least one of logic, sensors, memory, wireless capability, and telemetry.

13. The system of claim 8, wherein the retainer is rechargeable.

14. The system of claim 1, wherein the retainer is repeatedly removed and re-applied until osseointegration has occurred.

15. The system of claim 1, wherein the power source is removed after osseointegration has occurred.

16. The system of claim 1, wherein the power source is worn continually until osseointegration has occurred.

17. A method of alveolar ridge preparation, comprising
implanting a dental implant into the jawbone of a patient, the implant comprising an osteogenic screw configured for implant into a patient's jawbone, the screw being selectively anodized to form an electrically conductive first portion and an electrically insulated second portion, the electrically insulated second portion comprising about 75% of the surface of the screw and an electrical connector at a top portion of the screw configured to transmit power to the osteogenic screw;
operatively connecting the electrical connector to a removable power source; and
applying an electrical current for a treatment duration and treatment period such that osseointegration sufficient for placement of at least one of an abutment, artificial tooth, and a crown has occurred.

18. The method of claim 17, wherein the electrical current is about 1 μA-1 mA.

19. The method of claim 17, wherein the treatment duration comprises continuous treatment.

20. The method of claim 17, wherein the treatment period is about 0-6 months.

21. The method of claim 17, further comprising assessing the patient for eligibility for treatment.

22. The method of claim 17, further comprising confirming sufficient osseointegration has occurred.

23. The method of claim 17, further comprising placing at least one of an abutment, an artificial tooth, and a crown.

24. An osteogenic dental implant system, the system comprising:
an osteogenic screw configured for implant into a patient's jawbone, the screw being selectively anodized to form an electrically conductive first portion and an electrically insulated second portion, the electrically insulated second portion comprising about 75% of the surface of the screw; and
a dual purpose socket positioned at the top of the screw, the socket configured to attach to a removable electrical connector and attach to a mating mechanism for at least one of an abutment, an artificial tooth and a crown.

25. The system of claim 24, further comprising an electrical connector attached to the socket.

26. The system of claim 24, further comprising a mating mechanism attached to the socket.

27. The system of claim 24, wherein the electrically insulated portion comprises about 75% of the surface of the screw.

28. The system of claim 24, wherein the electrically conductive first portion comprises a titanium alloy.

29. The system of claim 24, wherein the electrically insulated second portion comprises a titanium dioxide.

30. The system of claim 24, wherein the electrically conductive first portion is on a thread portion of the screw.

31. The system of claim 24, wherein the electrically insulated second portion is on a thread portion of the screw.

32. The system of claim 24, wherein the removable power source comprises a temporary crown, an artificial tooth, or an abutment.

33. The system of claim 24, wherein the removable power source comprises a return electrode.

34. The system of claim 24, wherein the removable power source comprises a retainer that can be worn and removed by the patient.

35. The system of claim 34, wherein the retainer comprises a return electrode facing an inner portion of the patient's cheek.

36. The system of claim 34, wherein a large portion 75% of the retainer is electrically conductive.

37. The system of claim 34, wherein the retainer comprises at least one of logic, sensors, and memory.

38. The system of claim 34, wherein the retainer is rechargeable.

39. The system of claim 24, wherein the power source is removed after osseointegration has occurred.

40. The system of claim 24 wherein the power source is worn continually until osseointegration has occurred.

41. The system of claim 24, wherein the power source is repeatedly removed and re-applied until osseointegration has occurred.
